(19) United States
(12) Patent Application Publication
(10) Pub. No.: US 2017/0290844 A1
(43) Pub. Date: Oct. 12, 2017

(54) Methods of Treatment

(71) Applicant: GlaxoSmithKline Intellectual Property Development Limited, Brentford, Middlesex (GB)

(72) Inventors: Soren BEINKE, Stevenage, Hertfordshire (GB); Nikolai Nikolaevich BELYAEV, Stevenage, Hertfordshire (GB); Edith HESSEL, Stevenage, Hertfordshire (GB)

(21) Appl. No.: 15/511,678

(22) PCT Filed: Sep. 22, 2015
(86) PCT No.: PCT/EP2015/071638

§ 371 Date: Mar. 16, 2017

(30) Foreign Application Priority Data
Sep. 24, 2014 (GB) 1416832.2

(51) Int. Cl. A61K 31/5513; G01N 33/50; C12Q 1/68; C07K 16/28; A61K 31/55

(52) U.S. Cl. CPC A61K 31/5513; C07K 16/28; A61K 31/55; C12Q 1/6883; G01N 33/5041; C07K 2317/76; C12Q 2600/136

(57) ABSTRACT

The present invention relates to methods of treating or preventing respiratory infections, in particular respiratory infections in patients with an underlying respiratory disorder such as chronic obstructive pulmonary disease (COPD). The present invention also relates to methods of treating or preventing COPD or COPD exacerbations. The invention particularly describes the role of Notch 3 and/or Notch 4 signalling in such methods and the use of Notch 3 and/or Notch 4 as therapeutic and screening targets.

### FIELD OF THE INVENTION

The present invention relates to novel methods of treatment. More particularly, in one aspect, the present invention relates to methods of treating or preventing respiratory infections, in particular respiratory infections in patients with an underlying respiratory disorder such as chronic obstructive pulmonary disease (COPD). The present invention also relates to methods of treating or preventing COPD or COPD exacerbations. The invention particularly describes the role of Notch 3 and/or Notch 4 signalling in such methods and the use of Notch 3 and/or Notch 4 as therapeutic and screening targets. In another aspect, the invention relates to pharmaceutical compositions for use in such methods of treatment comprising an inhibitor of Notch 3 and/or Notch 4 signalling. In a further aspect, the invention relates to methods of screening for inhibitors of Notch 3 and/or Notch 4 signalling useful in said methods of treatment.

### BACKGROUND OF THE INVENTION

Notch signalling (FIG. 1) is an evolutionary conserved pathway that was originally discovered in Drosophila melanogaster and has been shown to influence cell-fate decisions in many different organisms. In mammals, there are 4 Notch receptors (Notch 1-4) which can bind ligands of the Jagged (Jagged 1 and 2) and the Delta-like ligand (DLL1 3 and 4) families. Notch receptors are type I trans-membrane proteins and upon ligand engagement proteolytic cleavage events liberate the intracellular domain of Notch (NICD) which then translocates to the nucleus where it binds the helix-loop-helix transcription factor CSL/RBP-J. In the absence of NICD, CSL/RBP-J forms a repressor complex thus inhibiting transcription, whereas upon interaction with NICD, co-repressors are displaced and co-activators are recruited, leading to transcriptional activation of target genes.

As described above, proteolytic cleavage events targeting Notch receptors are absolutely crucial for successful signal transduction. These are mediated by the ADAM family of proteases and the multi-protein γ-secretase complex. Inhibition of γ-secretase complex activity has been used to block Notch signalling therapeutically and as a tool to study the biological functions of this pathway.

Viral and bacterial respiratory infections are key triggers of COPD exacerbations which are an acute worsening of symptoms associated with excessive mucus production and neutrophilic inflammation in the airways. The airway epithelium plays a critical role in the innate defence against respiratory pathogens by providing a mucociliary escalator to expel pathogens and coordinate the immune response via the expression of inflammatory mediators. Both of these epithelial cell functions have been shown to be dysfunctional in COPD.

Notch signalling has been implicated in cell fate determination in large airway epithelial cells by promoting differentiation of basal cells towards the mucus producing goblet cell lineage under physiological conditions (Guseh J S et. al., Development, May 2009; Rock J R et. al., Cell Stem Cells, May 2011).

Currently no effective treatments are available that concurrently treat neutrophilic inflammation and excessive mucus production in COPD exacerbations. CXCR2 inhibitors may be effective in reducing neutrophilic inflammation without any effect on the mucociliary responses, whereas mucolytics are generally not very effective and do not have any impact on inflammation. Furthermore, these treatments would not have any long term impact on the restoration of the epithelium.

The present inventors have found that inhibition of Notch 3 and/or Notch 4 signalling, for example during a viral respiratory infection-induced exacerbation in COPD, may result in simultaneous reduction of neutrophilic inflammation and mucus production while leaving essential immune responses intact. It is believed that this may alleviate pathology, restore the mucociliary escalator and homeostasis of the lung, thus reducing the risk of secondary bacterial infections.

### SUMMARY OF THE INVENTION

Thus, in one aspect, there is provided a method of treating or preventing a respiratory infection which comprises inhibiting Notch 3 and/or Notch 4 signalling in a mammal.

In another aspect, there is provided a method of treating or preventing COPD which comprises inhibiting Notch 3 and/or Notch 4 signalling in a mammal.

In another aspect, there is provided a method of treating or preventing a COPD exacerbation which comprises inhibiting Notch 3 and/or Notch 4 signalling in a mammal.

In another aspect, there is provided a pharmaceutical composition for use in the treatment or prevention of a respiratory infection, COPD or a COPD exacerbation comprising an inhibitor of Notch 3 and/or Notch 4 signalling, together with at least one pharmaceutical carrier, diluent or excipient.

In a further aspect, there is provided a method of screening for an inhibitor of Notch 3 and/or Notch 4 signalling for use in treating or preventing respiratory infections, COPD or COPD exacerbations comprising the step of determining whether an agent inhibits the Notch 3 and/or Notch 4 pathway.

### DESCRIPTION OF THE DRAWINGS

FIG. 1 Notch signaling pathway

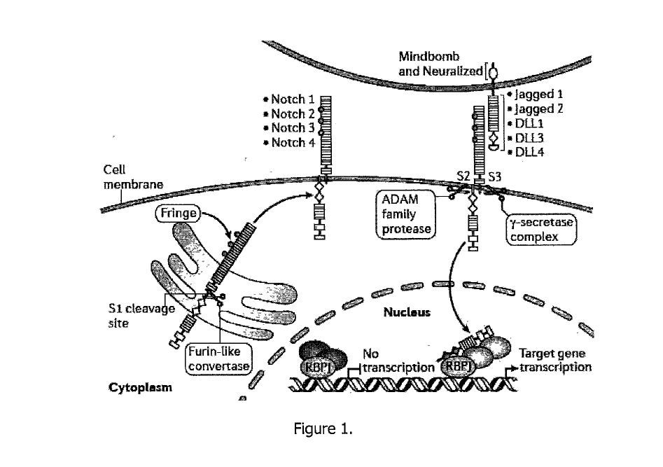

FIG. 2 Gene expression analysis of bronchial brushings obtained from infrequently and frequently exacerbating ex-smoker COPD patients. Notch 3 as well as Notch target genes are elevated in frequently exacerbating as compared to infrequently exacerbating COPD patients.

FIG. 3 Genome-wide transcriptional analysis by microarray of COPD bronchial epithelial cells infected for 72 h with HRV16 and treated with γ-secretase inhibitor CompA. Inhibition of Notch signalling restores cilia associated genes and inhibits upregulation of goblet cell associated genes after HRV16 infection. Interestingly, the interferon response is left unaltered. (A) Principal component analysis (PCA) illustrating two components that best resolve epithelial cell transcriptional signatures based on treatment. (B) Venn diagram illustrating the magnitude of epithelial cell responses in COPD donors. Numbers indicate the number of probes that are regulated by each treatment. (C) Heatmap illustrating averaged gene expression in bronchial epithelial cells (BECs) from COPD infected with HRV16 alone or in combination with CompA.

FIG. 4 Analysis of gene expression in COPD BECs infected with HRV16 and treated with CompA or a Notch sparing γ-secretase modulator for 72 h. The Notch sparing γ-secretase modulator has no effect on the expression of Notch target genes or mucociliary end-points in HRV16 infected cells. (A) Bar-charts illustrating gene expression of Notch target genes in ALI cultures of COPD BECs by real-time PCR. (B) Bar-charts illustrating gene expression of ciliated and goblet cell associated genes in ALI cultures of COPD BECs by real-time PCR. Legend denotes concentration in nM.

FIG. 5 Mucociliary responses of COPD BECs infected with HRV16 and treated with CompA for 72 h. Inhibition of Notch signalling inhibits mucus production in HRV16 infected cells. (A) Bar-charts illustrating gene expression of goblet cell specific genes in ALI cultures of COPD BECs by real-time PCR. (B) Transverse sections of COPD BECs stained with AB/PAS illustrating mucus expression.

FIG. 6 Mucociliary responses of COPD BECs infected with HRV16 alone or in combination with CompA for 72 h. Inhibition of Notch signalling restores ciliation after HRV16 infection. (A) Bar-charts illustrating gene expression of ciliated cell specific genes in ALI cultures of COPD BECs by real-time PCR. (B) Representative micrographs illustrating immunofluorescence staining of β-tubulin IV expression in ALI cultured COPD BECs infected with HRV16 alone or in combination with CompA.

FIG. 7 Gene expression analysis of immune mediators by real-time PCR in COPD BECs 24 h post HRV16 infection with or without CompA treatment. Inhibition of Notch signalling leads to transcriptional inhibition of genes encoding neutrophilic chemokines, whereas gene expression of T-cell associated chemokines is less affected. (A) Gene expression of chemokines associated with neutrophilic inflammation. (B) Gene expression of chemokines associated with T-cell recruitment.

FIG. 8 Quantification of protein secretion of immune mediators in the supernatants of ALI cultured COPD BECs 72 h post infection with HRV16 alone or in combination with CompA. Inhibition of Notch signalling leads to reduced secretion of neutrophilic chemokines, whereas secretion of T-cell associated chemokines is less affected. (A) Protein concentration of chemokines associated with neutrophilic inflammation. (B) Protein concentration of chemokines associated with T-cell recruitment.

FIG. 9 Neutrophil chemotaxis is reduced in response to supernatants treated with CompA as compared to untreated infected samples. T-cell chemotaxis is unaltered. Analysis of healthy neutrophil (A) and T-cell (B) chemotaxis in response to supernatants collected from ALI cultured COPD BECs 72 h post HRV16 infection alone or in combination with CompA.

FIG. 10 Analysis of inflammation and mucus gene expression in the lungs of female Balb/c mice following HRV1B inoculation and treatment with γ-secretase inhibitor CompB. Recruitment of neutrophils and mucin gene expression is reduced in lungs of γ-secretase treated animals at 8 h post infection. (A) Neutrophil influx into the lungs (BAL) at 8 h post infection. (B) Gene expression of Muc5ac at 8 h post infection.

FIG. 11 Cell surface expression of CD66a/c/e (CEACAM1/5/6) on COPD BECs with and without GSI treatment under steady state. Representative histograms illustrating reduced CD66a/c/e expression on cells treated with GSI for 48 h.

FIG. 12 Expression of Notch receptors on ALI cultured COPD BECS by flow cytometry. Representative histograms illustrating cell surface expression of Notch 3 with lower levels of Notch 4 on COPD BECs under steady state. Notch 1 and 2 are not expressed.

FIG. 13 Expression of Notch receptors in COPD lungs by immunohistochemistry. (A) Representative micrographs illustrating expression of Notch 3 with lower levels of Notch 4 in COPD lungs at stable state. Notch 1 is not expressed. (B) Quantification of receptor expression in different cell populations found in the lung.

### DETAILED DESCRIPTION OF THE INVENTION

According to the present invention it has been found that inhibition of Notch 3 and/or Notch 4 signalling may result in the simultaneous reduction of neutrophilic inflammation and mucus production. Thus, in one embodiment, the present invention provides a method of treating or preventing neutrophilic inflammation and mucus production which comprises inhibiting Notch 3 and/or Notch 4 signalling in a mammal. In another embodiment, the present invention provides a method of treating or preventing neutrophilic inflammation which comprises inhibiting Notch 3 and/or Notch 4 signalling in a mammal. In a further embodiment, the present invention provides a method of treating or preventing mucus production which comprises inhibiting Notch 3 and/or Notch 4 signalling in a mammal.

In one aspect, the present invention provides a method of treating or preventing a respiratory infection which comprises inhibiting Notch 3 and/or Notch 4 signalling in a mammal.

Respiratory infections which may be treated or prevented include viral infections, bacterial infections and/or infections with other pathogens.

In one embodiment, the respiratory infection is a viral infection. Viral infections include, for example, infections by influenza, rhinovirus, respiratory syncytial virus (RSV), human parainfluenza virus (HPIV), adenovirus and/or coronavirus.

In another embodiment, the respiratory infection is a bacterial infection. Bacterial infections include, for example, infections by S. Pneumoniae, H. Influenzae, and/or M. Catarrhalis. The bacterial infection may be secondary to a viral infection.

In a further embodiment, the respiratory infection is an infection with another pathogen, for example, aspergillosis and/or leishmaniasis.

The present invention also provides a method of treating or preventing a respiratory infection which comprises inhibiting Notch 3 and/or Notch 4 signalling in a mammal with an underlying respiratory disorder. An underlying respiratory disorder may be, for example, COPD, asthma, cystic fibrosis, acute respiratory distress syndrome (ARDS) and/or idiopathic pulmonary fibrosis (IPF). In one embodiment, the underlying disorder is COPD. In a further embodiment, the underlying disorder is asthma.

Thus, in one embodiment, the present invention provides a method of treating or preventing a respiratory infection which comprises inhibiting Notch 3 and/or Notch 4 signalling in a mammal with COPD. In another embodiment, the present invention provides a method of treating or preventing a respiratory infection which comprises inhibiting Notch 3 and/or Notch 4 signalling in a human with COPD. In another embodiment, the present invention provides a method of treating or preventing a viral respiratory infection which comprises inhibiting Notch 3 and/or Notch 4 signalling in a human with COPD. In another embodiment, the present invention provides a method of treating or preventing a bacterial respiratory infection which comprises inhibiting Notch 3 and/or Notch 4 signalling in a human with COPD. In a further embodiment, the present invention provides a method of treating or preventing a secondary bacterial respiratory infection which comprises inhibiting Notch 3 and/or Notch 4 signalling in a human with COPD.

In view of the role of Notch 3 and/or Notch 4 signalling in neutrophilic inflammation and mucus production, in addition to the use of a Notch 3 and/or Notch 4 signalling inhibitor in treating or preventing a respiratory infection in patients with COPD, it is believed that such inhibitors may be useful in treating or preventing the underlying COPD. Thus, in one embodiment, the present invention provides a method of treating or preventing COPD which comprises inhibiting Notch 3 and/or Notch 4 signalling in a mammal.

Although COPD exacerbations are generally triggered by a respiratory infection, a significant proportion of exacerbations may also be triggered by, for example, air pollutants, the patient's own neutrophils or an unidentifiable cause. It is believed that a Notch 3 and/or Notch 4 signalling inhibitor may be useful in treating or preventing COPD exacerbations irrespective of the cause. Thus, in one embodiment, the present invention provides a method of treating or preventing a COPD exacerbation which comprises inhibiting Notch 3 and/or Notch 4 signalling in a mammal.

Certain COPD patients are referred to as “frequent exacerbators” due to their increased susceptibility to exacerbations. Such patients typically suffer from accelerated decrease in lung function, a poorer quality of life and a higher risk of hospitalisation and death. Thus, in one embodiment, the present invention provides a method of treating or preventing a respiratory infection which comprises inhibiting Notch 3 and/or Notch 4 signalling in a COPD patient who is a frequent exacerbator. In another embodiment, the present invention provides a method of treating COPD which comprises inhibiting Notch 3 and/or Notch 4 signalling in a COPD patient who is a frequent exacerbator. In a further embodiment, the present invention provides a method of treating or preventing a COPD exacerbation which comprises inhibiting Notch 3 and/or Notch 4 signalling in a COPD patient who is a frequent exacerbator.

Notch 3 and/or Notch 4 signalling may be inhibited by any suitable inhibitor. As used herein, the term “inhibitor” can be any agent capable of inhibiting Notch 3 and/or Notch 4 signalling, i.e. any compound or treatment that interrupts the receptor:ligand interaction or any other signalling event downstream of the Notch 3 and/or Notch 4 receptor. In one embodiment, the inhibitor is a γ-secretase inhibitor.

The inhibitor may be of varied nature and origin including natural origin (e.g. plant, animal, eukaryatic, bacterial, viral) or synthetic (e.g. an organic, inorganic, synthetic or semi-synthetic molecule). For example, the inhibitor may be a nucleic acid, a peptide, a polypeptide, a protein or a chemical compound.

In one embodiment the inhibitor may be an antisense nucleic acid capable of inhibiting expression of Notch 3 and/or Notch 4. The antisense nucleic acid can comprise all or part of the sequence of the Notch 3 and/or Notch 4 receptor, or of a sequence that is complementary thereto. The antisense sequence can be a DNA, an RNA (e.g. siRNA), a ribozyme, etc. It may be single-stranded or double stranded. It can also be an RNA encoded by an antisense gene. When an antisense nucleic acid comprising part of the sequence of the gene or messenger RNA under consideration is being used, it is preferred to use a part comprising at least 10 consecutive bases from the sequence, more preferably at least 15, in order to ensure specific hybridisation. In the case of an antisense oligonucleotide, it typically comprises less than 100 bases, for example in the order of 10 to 50 bases. This oligonucleotide can be modified to improve its stability, its nuclease resistance, its cell penetration, etc. Perfect complementarily between the sequence of the antisense molecule and that of the target gene or messenger RNA is not required, but is generally preferred.

According to another embodiment, the inhibitor compound may be a polypeptide. It may be, for example, a peptide comprising a region of the Notch 3 and/or Notch 4 receptor, and capable of antagonising its activity. A peptide advantageously comprises from 5 to 50 consecutive amino acids of the primary sequence of Notch 3 and/or Notch 4 receptor under consideration, typically from 7 to 40. The polypeptide may also be an antibody against the Notch 3 and/or Notch 4 receptor.

The term “antibody” is used herein to refer to molecules with an immunoglobulin-like domain (for example IgG, IgM, IgA, IgD or IgE) and includes monoclonal, recombinant, polyclonal, chimeric, human, humanised, multi-specific antibodies, including bispecific antibodies, and heteroconjugate antibodies; a single variable domain (e.g., VH, VHH, VL, domain antibody (dAb™)), antigen binding antibody fragments, Fab, F(ab′)2, Fv, disulphide linked Fv, single chain Fv, disulphide-linked scFv, diabodies, TANDABS™, etc. and modified versions of any of the foregoing (for a summary of alternative “antibody” formats see Holliger and Hudson, Nature Biotechnology, 2005, Vol 23, No. 9, 1126-1136). Alternative antibody formats include alternative scaffolds in which one or more CDRs can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. Further antibodies include mAbdAbs, dAb™ conjugates and dAb™ fusions.

The term “domain” refers to a folded protein structure which retains its tertiary structure independent of the rest of the protein. Generally domains are responsible for discrete functional properties of proteins and in many cases may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as rodent (for example, as disclosed in WO 00/29004), nurse shark and Camelid VHH dAbs™. Camelid VHH are immunoglobulin single variable domains that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains.

As used herein, the term “mAbdAb” refers to a monoclonal antibody linked to a further binding domain, in particular a single variable domain such as a domain antibody. A mAbdAb has at least two antigen binding sites, at least one of which is from a domain antibody, and at least one is from a paired VH/VL domain. mAbdAbs are described in WO2009/068649.

A “dAb™ conjugate” refers to a composition comprising a dAb to which a drug is chemically conjugated by means of a covalent or noncovalent linkage. Preferably, the dAb and the drug are covalently bonded. Such covalent linkage could be through a peptide bond or other means such as via a modified side chain. The noncovalent bonding may be direct (e.g., electrostatic interaction, hydrophobic interaction) or indirect (e.g., through noncovalent binding of complementary binding partners (e.g., biotin and avidin), wherein one partner is covalently bonded to drug and the complementary binding partner is covalently bonded to the dAb™). When complementary binding partners are employed, one of the binding partners can be covalently bonded to the drug directly or through a suitable linker moiety, and the complementary binding partner can be covalently bonded to the dAb™ directly or through a suitable linker moiety.

As used herein, “dAb™ fusion” refers to a fusion protein that comprises a dAb™ and a polypeptide drug (which could be a dAb™ or mAb). The dAb™ and the polypeptide drug are present as discrete parts (moieties) of a single continuous polypeptide chain.

Such antibodies, fragments, or derivatives can be produced by conventional techniques comprising immunising an animal and recovering the serum (polyclonal) or spleen cells (in order to produce hybridomas by fusion with appropriate cell lines).

Methods for producing polyclonal antibodies in various species are described in the prior art. Typically, the antigen is combined with an adjuvant (e.g. Freund's adjuvant) and administered to an animal, typically by subcutaneous injection. Repeated injections can be performed. Blood samples are collected and the immunoglobulin or serum is separated. Conventional methods for producing monoclonal antibodies comprise immunising of an animal with an antigen, followed by recovery of spleen cells, which are then fused with immortalised cells, such as myeloma cells. The resulting hybridomas produce monoclonal antibodies and can be selected by limiting dilution in order to isolate individual clones. Fab or F(ab′)2 fragments can be produced by protease digestion, according to conventional techniques.

Antibodies which inhibit Notch 3 and/or Notch 4 are known in the art.

According to further embodiment, the inhibitor may be a chemical compound, of natural or synthetic origin, particularly an organic or inorganic molecule, capable of modulating the expression or the activity of the Notch 3 and/or Notch 4 receptor. In one embodiment, the chemical compound is a γ-secretase inhibitor. In another embodiment, the chemical compound is (S)-2-[2-(3,5-difluorophenyl)-acetylamino]-N—((S)-1-methyl-2-oxo-5-phenyl-2,3-dihydro-1H-benzo[e][1,4]diazepin-3-yl)-propionamide. In a further embodiment, the chemical compound is N2-[(2S)-2-(3,5-difluorophenyl)-2-hydroxyethanoyl]-N1-[(7S)-5-methyl-6-oxo-6,7-dihydro-5H-di benzo[b,d]azepin-7-yl]-L-alaninamide.

In one embodiment of the invention, Notch 3 signalling is inhibited. In another embodiment, Notch 4 signalling is inhibited. In a further embodiment, Notch 3 and Notch 4 signalling are inhibited.

According to the invention, the inhibitors of Notch 3 and/or Notch 4 signalling are typically administered to a subject in a therapeutically effective amount. As used herein, the “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function.

As used herein, “treating” or “treatment” in reference to a disorder means: (1) to ameliorate or prevent the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As used herein, “preventing” or “prevention” in reference to a disorder means the prophylactic administration of a drug to substantially diminish the likelihood or severity of a disorder or biological manifestation thereof, or to delay the onset of such disorder or biological manifestation thereof.

In one embodiment, the invention relates to the treatment of a disorder. In a further embodiment, the invention relates to the prevention of a disorder.

While it is possible that, for use in therapy, the inhibitor may be administered as the raw chemical, it is possible to present the active ingredient as a pharmaceutical composition. Accordingly, in another aspect, the invention provides a pharmaceutical composition for use in the treatment or prevention of a respiratory infection, COPD or a COPD exacerbation comprising an inhibitor of Notch 3 and/or Notch 4 signalling, together with at least one pharmaceutical carrier, diluent or excipient. The carrier(s), diluents(s) or excipient(s) must be acceptable in the sense of being compatible with the other ingredients of the composition and not deleterious to the recipient thereof. In accordance with another embodiment of the invention there is also provided a process for the preparation of a pharmaceutical composition including the inhibitor, or a pharmaceutically acceptable salt thereof, with at least one pharmaceutically acceptable carrier, diluent or excipient. The pharmaceutical composition can be for use in the treatment or prevention of any of the conditions described herein.

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered once or more than once a day. Such pharmaceutical compositions may be prepared by any of the methods well known in the pharmacy art.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s). In one embodiment, the pharmaceutical composition according to the invention is administered by the inhaled route.

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by reducing the compound to a suitable fine size and mixing with a similarly prepared pharmaceutical carrier such as an edible carbohydrate, as, for example, starch or mannitol. Flavouring, preservative, dispersing and colouring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and colouring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of the invention may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

Pharmaceutical compositions adapted for topical administration in the mouth include lozenges, pastilles and mouth washes.

Dosage forms for nasal or inhaled administration may conveniently be formulated as aerosols, solutions, suspensions drops, gels or dry powders.

For compositions suitable and/or adapted for inhaled administration, it is preferred that the agent is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 10 microns (for example as measured using laser diffraction). Compositions adapted for administration by inhalation include the particle dusts or mists. Suitable compositions wherein the carrier is a liquid for administration as a nasal spray or drops include aqueous or oil solutions/suspensions of the active ingredient which may be generated by means of various types of metered dose pressurised aerosols, nebulizers or insufflators.

Aerosol formulations, e.g. for inhaled administration, can comprise a solution or fine suspension of the agent in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, the pharmaceutical composition may be a dry powder inhalable composition. Such a composition can comprise a powder base such as lactose, glucose, trehalose, mannitol or starch, the agent, (preferably in particle-size-reduced form, e.g. in micronised form), and optionally a performance modifier such as L-leucine or another amino acid, cellobiose octaacetate and/or metals salts of stearic acid such as magnesium or calcium stearate.

Aerosol formulations are preferably arranged so that each metered dose or “puff” of aerosol contains a particular amount of a compound of the invention. Administration may be once daily or several times daily, for example 2, 3 4 or 8 times, giving for example 1, 2 or 3 doses each time. The overall daily dose and the metered dose delivered by capsules and cartridges in an inhaler or insufflator will generally be double those with aerosol formulations.

Pharmaceutical compositions adapted for parental administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

Antisense or RNA interference molecules may be administered to the mammal in need thereof. Alternatively, constructs including the same may be administered. Such molecules and constructs can be used to interfere with the expression of the protein of interest, e.g., Notch 3 and/or Notch 4 and as such, modify gene expression. Typically delivery is by means known in the art.

Antisense or RNA interference molecules can be delivered in vitro to cells or in vivo, e.g., to tumors of a mammal. Nodes of delivery can be used without limitations, including: intravenous, intramuscular, intraperitoncal, intra-arterial, local delivery during surgery, endoscopic, subcutaneous, and per os. Vectors can be selected for desirable properties for any particular application. Vectors can be viral or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

A therapeutically effective amount of the agent will depend upon a number of factors including, for example, the age and weight of the subject, the precise condition requiring treatment and its severity, the nature of the formulation, and the route of administration, and will ultimately be at the discretion of the attendant physician or veterinarian. In particular, the subject to be treated is a mammal, particularly a human.

The inhibitor may be administered in a daily dose. This amount may be given in a single dose per day or more usually in a number (such as two, three, four, five or six) of sub-doses per day such that the total daily dose is the same.

The inhibitor may be employed alone or in combination with other therapeutic agents.

The inhibitor for use according to the invention may be used in combination with or include one or more other therapeutic agents and may be administered either sequentially or simultaneously by any convenient route in separate or combined pharmaceutical compositions.

The inhibitor and pharmaceutical compositions for use according to the invention may be used in combination with or include one or more other therapeutic agents, for example selected from an anti-inflammatory agent such as a corticosteroid, an NSAID, a PI3Kd inhibitor, a PDE4 inhibitor or a non-steroidal GR agonist; a bronchodilator such as an anticholinergic agent or a β₂-adrenoreceptor agonist; an anti-muscarinic; an antiinfective agent such as an antibiotic or an antiviral; or an antihistamine.

In a further aspect, the present invention provides a method of screening for an inhibitor of Notch 3 and/or Notch 4 signalling for use in treating or preventing respiratory infections, COPD or COPD exacerbations comprising the step of determining whether an agent inhibits the Notch 3 and/or Notch 4 pathway.

The present invention proposes, for the first time that Notch 3 and/or Notch 4 signalling is a potential therapeutic target for the treatment or prevention of respiratory infections, COPD or COPD exacerbations. Thus, the present invention provides a new target for the identification, validation, selection and optimisation of active agents on the basis of their ability to inhibit Notch 3 and/or Notch 4 signalling.

The present invention thus pertains to a method of identifying, screening, characterising or defining an agent which is capable of inhibiting Notch 3 and/or Notch 4 signalling for use in treating or preventing respiratory infections, COPD or COPD exacerbations. The methods can be used for screening for example large numbers of candidate compounds for clinical use in respiratory infections, COPD or COPD exacerbations.

The assays (screening methods) may be performed in a cell-based system, an animal system or by a cell free system. Such techniques will be apparent to a person skilled in the art and may be based on a measure of interaction (e.g. binding, displacement or competition assays) or a measure of a function of activity, transcription and the like.

Thus, for example, the present invention provides a method of testing the ability of an agent to inhibit Notch 3 and/or Notch 4 signalling.

Provided herein are screening methods for identifying inhibitors of Notch 3 and/or Notch 4 signalling as being potentially useful in the treatment or prevention of respiratory infections, COPD or COPD exacerbations. One method involves screening for an inhibitor of Notch 3 and/or Notch 4 signalling, including the steps of contacting a peptide, which may be modified by acetylation, with a Notch 3 and/or Notch 4 receptor or a fragment thereof in the presence and in the absence of a test substance, and identifying a test substance as an inhibitor of Notch 3 and/or Notch 4 activity. Test agents (or substances) for screening as inhibitors of Notch 3 and/or Notch 4 can be from any source known in the art. They can be natural products, purified or mixtures, synthetic compounds, members of compound libraries, etc. The test substances can be selected from those that have been identified previously to have biological or drug activity or from those that have not.

In a further embodiment the method of screening for an inhibitor of Notch 3 and/or Notch 4 receptor includes a binding assay. Thus a compound which inhibits the binding of the Notch 3 and/or Notch 4 receptor to its substrate can be identified in competition or direct binding assays. Both direct and competition binding assay formats are similar to the formats used in immunoassays and receptor binding assays and will be generally known to a person skilled in the art.

The following examples are set forth to illustrate the effectiveness of the approach described in the present invention and to further exemplify particular applications of general processes described above. Accordingly, the following Example section is in no way intended to limit the scope of the invention contemplated herein.

### Examples

Materials and Methods:

Cell and Tissue Preparation

Air-liquid interface (ALI) cultured human bronchial epithelial cells (BECs) from chronic obstructive pulmonary disease (COPD) donors were either bought fully differentiated (Epithelix Sarl, Cat. No. EP10MD) or cultured in-house using the Pneumocult method (StemCell Technologies, Cat. No. 05001), in a 24-well format. Primary cells for in-house ALI cultures were obtained from Epithelix Sarl, Cat. No. EP18AB.

Bronchial brushings were obtained from frequently (more than 2 a year) or infrequently (0 a year) exacerbating COPD patients by experimental bronchoscopies carried out at the University of Manchester Experimental Medicines Unit. Bronchial brushings were stored in TRIZOl™ reagent (Life Technologies, Cat. No. 15596-026), for downstream transcriptional analyses.

For in-vivo experiments, 8 weeks old female BALB/c mice were intra-nasally infected with 1×10⁸ plaque forming units (PFUs) of human rhinovirus serotype 1b (HRV1b) virus in a total of 50 μl and either treated or not with a γ-secretase inhibitor CompB N2-[(2S)-2-(3,5-difluorophenyl)-2-hydroxyethanoyl]-N1-[(7S)-5-methyl-6-oxo-6,7-dihydro-5H-dibenzo[b,d]azepin-7-yl]-L-alaninamide at a concentration of 0.5 mg/kg. To assess inflammation to the lung, broncho-alveolar lavage fluid (BALF) was collected and cellular infiltrates analysed on the Sysmex cell analysis system (Milton Keynes, UK). Subsequently, whole lung was placed into 1 ml of TRIZOL™ reagent for downstream gene expression analysis.

Rhinoviral Infection of ALI Cultured COPD BECs

Prior to infection, ALI cells were washed twice in warm PBS (with Ca₂⁺/Mg₂⁺). Subsequently, human rhinovirus 16 was applied at multiplicity of infection (MOI) of 1 or 5 in a total volume of 50 μl culture medium. The virus was allowed to attach for 1h at room temperature with gentle agitation. Consequently, the virus was washed off with warm PBS (with Ca₂⁺/Mg₂⁺) and cells were further cultured at 37° C. for the indicated amount of time either with or without the addition of a rsecretase inhibitor CompA (S)-2-[2-(3,5-difluorophenyl)-acetylamino]-N—((S)-1-methyl-2-oxo-5-phenyl-2,3-dihydro-1H-benzo[e][1,4]diazepin-3-yl)-propionamide, (Calbiochem Cat. No. 565790).

Analysis of Gene Expression

Ribonucleic acid (RNA) from ALI cultured COPD BECs was prepared by lysing the cells in RLT buffer supplied with Promega Total RNA Isolation System (Promega, Cat. No. Z3505). RNA was then extracted by using the manufacturer's protocol with automation. Murine total lung RNA was isolated by first homogenisation of the lung in 1 ml TRIZOL™ reagent, then subsequent phase separation with 1-bromo-3-chloropropane (BCP) (MRC, Cat. No. BP151). The aqueous phase was then mixed with 70% ethanol and final RNA extraction was achieved using the Promega Total RNA Isolation System with automation. Complementary deoxyribonucleic acid (cDNA) was synthesised by reverse transcription using MultiScribe™ Reverse Transcriptase and random primers according to the manufacturer's protocol (Applied Biosystems, Cat. No. 4368814). Amplification of specific targets was performed by quantitative real-time polymerase chain reaction (qRT-PCR) using the TaqMan® gene expression system (Applied Biosystems Cat. No. 4369510) and ready to use TaqMan® primer/probes (see Table 1). Global gene expression analysis was performed by microarray using the Human Genome U133 Plus 2.0 genechip and was done either by Epistem Ltd. (Manchester, UK) or Expression Analysis, Inc. (Durham, US). Data analysis was performed using Array Studio software (OmicSoft Corporation, Cary, US) with GC-RMA normalisation algorithm. Probes with intensities less than the geometric mean of 64 were discarded. Subsequently, probes that fulfilled the criteria of at least 1.2 fold regulation (bronchial brushings data set) or 2 fold regulation (ALI cultures data set) between any condition were taken for further analysis. The final gene lists were generated by performing analysis of variance (ANOVA) with the cut-off filter of p≦0.05.

Analysis of Protein Expression

Detection of secreted proteins was done on the Meso Scale Discovery (MSD®) platform according to the manufacturer's protocol. The list of specific assays is provided in Table 2. Chemokines CXCL6 and CCL5 were detected by enzyme linked immunosorbent assay (ELISA) kits both bought from R&D systems, Cat. Nos. DY333 and DRN00B, respectively. Analysis of cell surface expression of proteins was performed by flow cytometry. Briefly, cells grown at the ALI were detached by StemPro Accutase treatment for 20 mins at 37° C. (Life Technologies, Cat. No. A1110501). Cell concentration was then adjusted to 4×10⁶ cells/ml and staining was performed with Alexa Fluor® 488 conjugated CD66a/c/e (clone ASL-32, Biolegend), Brilliant Violet® 421 conjugated CD271 (C40-1457, BD Pharmingen), allophycocyanin (APC) conjugated CD133 (clone AC133, Miltenyi Biotec) and APC-Cy7 conjugated CD56 (clone HCD56, Biolegend). All anti-Notch receptor antibodies were unconjugated and were therefore revealed by secondary staining with either phycoerythrin (PE) conjugated Streptavidin (Biolegend) or goat anti-rabbit antibody (Life Technologies). In some cases PE-Cy7 conjugated anti-mouse IgG1 was used (clone RMG1-1, Biolegend). Details of anti-Notch receptor antibodies are provided in Table 3. Analytical flow cytometry was performed using the FACSCanto II (Beckton Dickinson) and analysed using FlowJo software (Tree Star, Inc. Ashland, US).

Histology and Imaging

ALI cultured BECs were fixed in 4% paraformaldehyde (PFA) overnight. Mucus load was assessed by histological sections with Alcian Blue/Periodic Acid Schiff (AB/PAS) stainings and were performed by the GSK PTS histological core facility. To detect cilia, fixed membranes were stained with an anti-β-tubulin IV antibody (clone ONS.1A6, Sigma Aldrich) and revealed by donkey anti-mouse IgG Alexa Fluor® 488 antibody (Life Technologies). Images were captured on the Axioskop 2 Plus microscope made by Zeiss.

Immunohistochemistry

Lung biopsies were fixed in paraffin blocks and subsequently sectioned at 3 μm thickness on the RM2235 Microtome (Leica Biosystems). Slides were processed and stained on the BenchMark ULTRA platform (Ventana Medical Systems), and images were captured on the Axioskop 2 Plus microscope made by Zeiss. Details of antibodies used are provided in Table 4.

TABLE 1
TaqMan ® primer/probe assays used to analyse expression
of target genes in ALI cultured BECs or whole murine lungs.

| Target | TaqMan assay ID |
|---|---|
| SPDEF | Hs01026050_m1 |
| MUC5AC | Hs00873651_mH |
| MUC5B | Hs00861595_m1 |
| FOXJ1 | Hs00230964_m1 |
| CBE1 | Hs00375668_g1 |
| DNAI2 | Hs00224913_m1 |
| CXCL1 | Hs00236937_m1 |
| CXCL5 | Hs01099660_g1 |
| CXCL6 | Hs00605742_g1 |
| CXCL8 | Hs00174103_m1 |
| CXCL10 | Hs01124251_g1 |
| CXCL11 | Hs04187682_g1 |
| CCL5 | Hs00982282_m1 |
| Muc5ac | Mm01276718_m1 |

TABLE 2
Details of assays for protein detection on the MSD platform.

| | | Barcode No. | Barcode No. | Barcode No. | |
|---|---|---|---|---|---|
| Analyte | Catalog No. | Plate 1 | Plate 2 | Plate 3 | Kit Lot No. |
| Cxcl1 | N451A-1 | 25D7YA1020 | 25D7YAO012 | 25D7YAJ016 | Z0044946 |
| Cxcl5 | N45ZA-1 | 25D8AAM025 | 25D8AAW020 | 25D8AA7023 | Z00X2494 |
| Cxcl8 | K15001B-4 | 2CD05AE011 | 2CD05AZ082 | 2CD05AY095 | K0033878 |
| Cxcl10 | K15001B-4 | 2CD05AE011 | 2CD05AZ082 | 2CD05AY095 | K0033878 |
| Cxcl11 | K151AWC-2 | 26D1NAD949 | 26D1NAZ959 | 26D1NAI951 | K0034236 |

TABLE 3
Details of anti-Notch receptor antibodies used to evaluated
Notch receptor expression on ALI cultured BECs.

| Notch receptor | Manufacturer | Catalog No. | Clone |
|---|---|---|---|
| Notch 1 | Abcam | ab44986 | A6 |
| Notch 2 | Miltenyi Biotec | 130-096-980 | MHN2-25 |
| Notch 3 | Abcam | ab23426 | — |
| Notch 4 | Abcam | ab134831 | — |

TABLE 4
Details of anti-Notch receptor antibodies used to evaluated
Notch receptor expression in COPD lungs.

| Notch receptor | Manufacturer | Catalog No. | Clone |
|---|---|---|---|
| Notch 1 | Abcam | ab128076 | mN1A |
| Notch 3 | Abcam | ab23426 | — |
| Notch 4 | Abcam | ab134831 | — |

Results

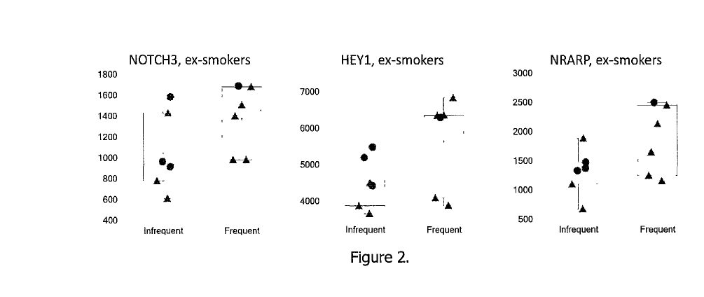

Components of the Notch pathway are differentially expressed in bronchial epithelial brushings from COPD patients suggesting that the Notch pathway and specifically the Notch 3 pathway, may play a role in the pathogenic phenotype of epithelial cells in COPD (FIG. 2).

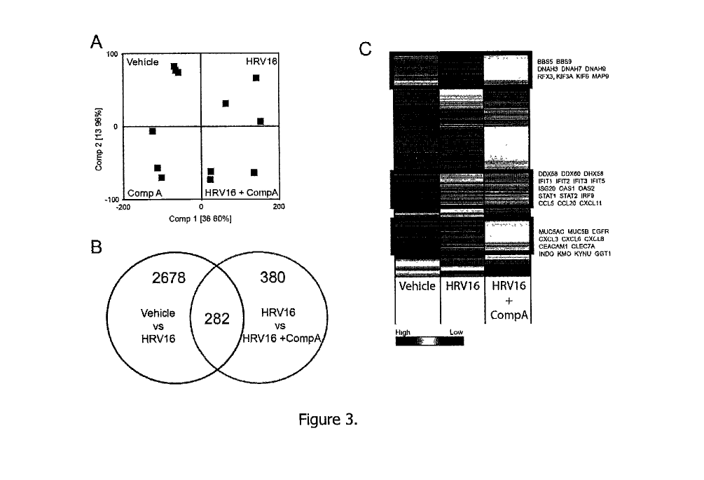

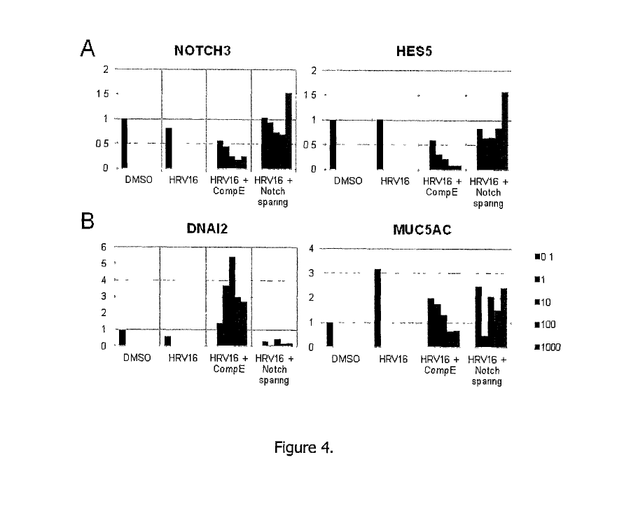

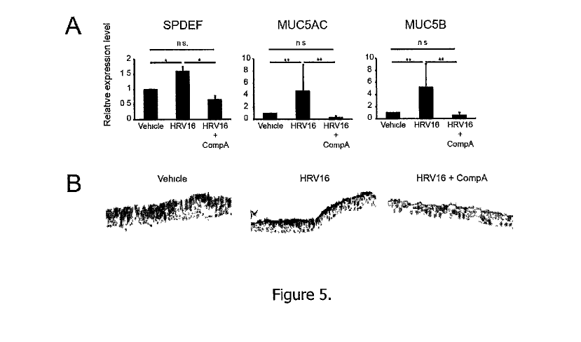

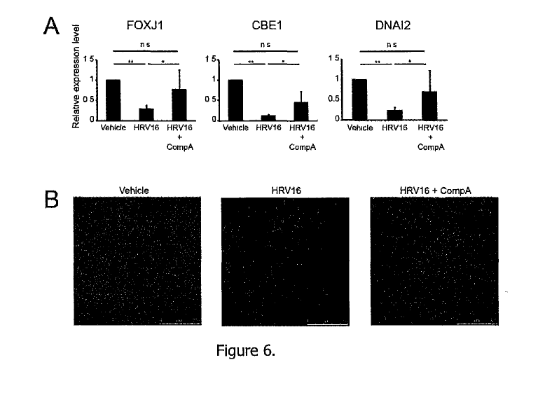

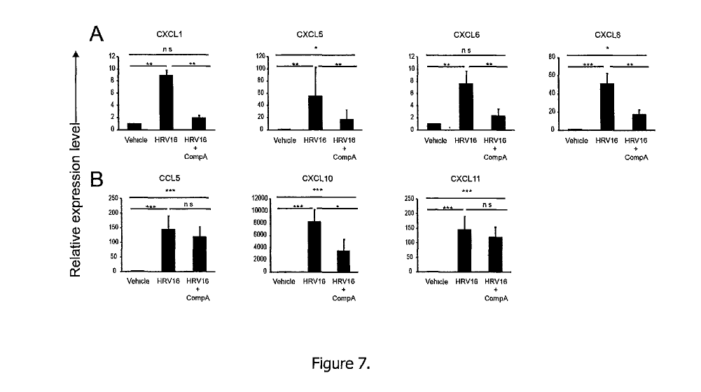

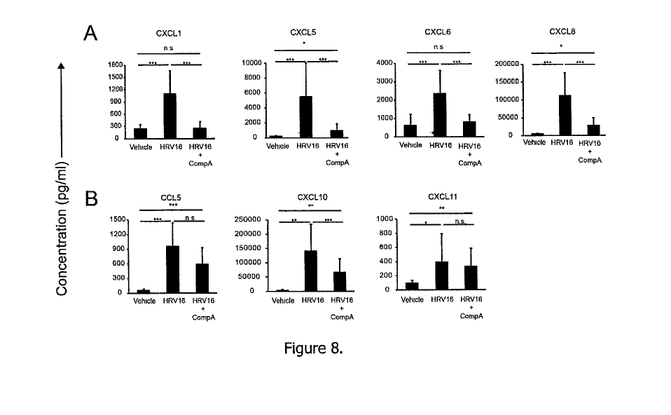

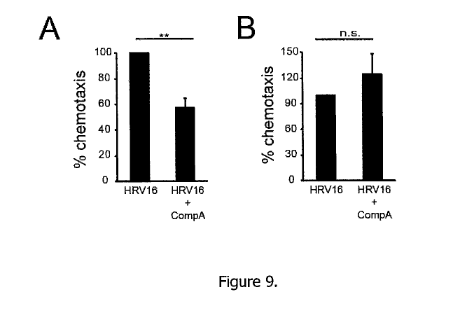

It has been demonstrated that interference with Notch signalling by γ-secretase inhibition (by treatment with CompA) reduces mucus secretion and prevents the loss of ciliation in healthy and COPD human bronchial epithelial cells (BECs) cultured at the air-liquid interface (ALI) and infected with human rhinovirus 16 (HRV16) (FIGS. 5 and 6). Furthermore, Notch inhibition reduces the HRV16 induced expression of neutrophila associated chemokines by these cells while T-cell chemokines and the anti-viral type 1 interferon response are left intact (FIGS. 3, 7 and 8). As a consequence, neutrophil but not T-cell chemotaxis is reduced in response to supernatants from Notch inhibited epithelial cells (FIG. 9). These effects are specific to the Notch pathway, since a Notch sparing γ-secretase modulator, which lacks activity against Notch receptors, did not inhibit Notch target genes and, furthermore, did not alter the mucociliary response after HRV16 infection, suggesting that modulation of ciliation and mucus production is regulated by Notch signalling (FIG. 4).

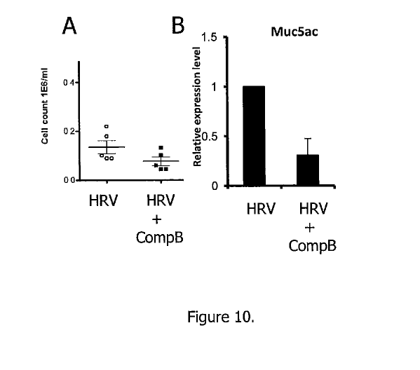

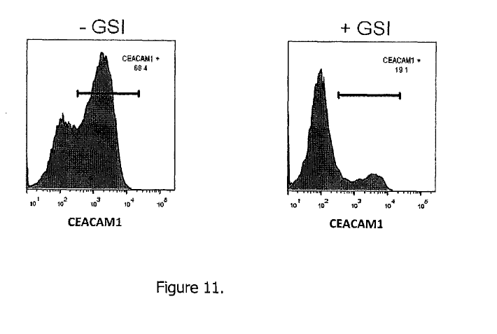

It has also been demonstrated that inhibition of Notch signalling reduces neutrophilia and mucin expression in the lungs of HRV1b infected mice in-vivo (FIG. 10). Also, Notch inhibition reduces the expression of bacterial adhesion receptors at the gene and protein levels in BECs in vitro, suggesting a potential impact on secondary bacterial infections (FIGS. 3 and 11).

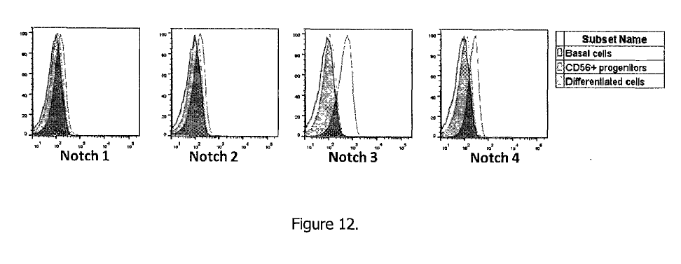

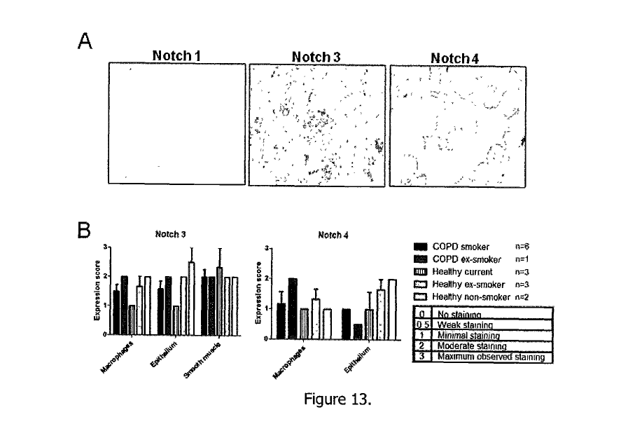

Finally, it has also been found that epithelial progenitor cells predominantly express Notch 3 receptors (FIG. 12) in vitro, mirroring expression in COPD lungs, where high expression of Notch 3 and, to a lesser extent, Notch 4 has been detected. Notch 1 and 2 have not been detected in vitro or in vivo (FIGS. 12, 13 and Human Protein Atlas Database: (http://www.proteinatlas.org/ENSG00000134250-NOTCH2/tissue). Furthermore, expression of NOTCH3 and other Notch target genes (HES5 and HEY1), are specifically regulated in response to Notch inhibition in vitro and in vivo.

Together, these data suggest that Notch 3 or Notch 4 or a combination of Notch 3 and 4 signalling specifically regulate pathologic mucociliary and inflammatory responses during viral infection of COPD airway epithelial cells and that Notch 3 or Notch 4 or a combination of Notch 3 and 4 inhibition may provide a potent therapeutic intervention to reduce these hallmarks of COPD exacerbations while leaving essential immune responses intact.

1. A method of treating or preventing a respiratory infection which comprises inhibiting Notch 3 and/or Notch 4 signalling in a mammal.

2. A method according to claim 1 wherein the respiratory infection is a viral infection.

3. A method according to claim 1 wherein the respiratory infection is a bacterial infection.

4. A method according to claim 1 wherein the respiratory infection is a secondary bacterial infection.

5. A method according to claim 1 wherein the mammal has an underlying respiratory disorder.

6. A method according to claim 5 wherein the disorder is COPD.

7. A method of treating or preventing COPD which comprises inhibiting Notch 3 and/or Notch 4 signalling in a mammal.

8. A method of treating or preventing a COPD exacerbation which comprises inhibiting Notch 3 and/or Notch 4 signalling in a mammal.

9. A method according to claim 1 wherein the Notch 3 and/or Notch 4 signalling is inhibited by an antibody or a chemical compound.

10. A method according to claim 1 wherein the mammal is a human.

11-13. (canceled)

14. A method of screening for an inhibitor of Notch 3 and/or Notch 4 signalling for use in treating or preventing respiratory infections, COPD or COPD exacerbations comprising the step of determining whether an agent inhibits the Notch 3 and/or Notch 4 pathway.
